(19) United States
(12) Patent Application Publication
(10) Pub. No.: US 2012/0302883 A1
(43) Pub. Date: Nov. 29, 2012

(54) METHOD AND SYSTEM FOR TREATMENT AND DIAGNOSIS USING ULTRASOUND

(75) Inventors: Dong-geon KONG, Yongin-si (KR); Joon-kee Cho, Yongin-si (KR); Ki-wan Choi, Anyang-si (KR)

(73) Assignee: SAMSUNG ELECTRONICS CO., LTD., Suwon-si (KR)

(21) Appl. No.: 13/470,897

(22) Filed: May 14, 2012

(30) Foreign Application Priority Data
May 25, 2011 (KR) 10-2011-0049801

(51) Int. Cl. A61B 8/08; A61B 8/14; A61N 7/00

(52) U.S. Cl. 600/439

(57) ABSTRACT

A method and system for treatment and diagnosis using ultrasound includes irradiating ultrasound for treatment into a treatment site, inducing a shear wave around the treatment site, irradiating ultrasound for diagnosis into the treatment site, and determining a degree of treatment or necrosis of a tissue of the treatment site by using a measured displacement of the shear wave.

### CROSS-REFERENCE TO RELATED APPLICATIONS

This application claims the priority benefit of Korean Patent Application No. 10-2011-0049801, filed on May 25, 2011, in the Korean Intellectual Property Office, the disclosures of which are incorporated herein in their entirety by reference.

### BACKGROUND

1. Field

The following description relates to methods and systems for treatment and diagnosis using ultrasound.

2. Description of the Related Art

With advances in medical technology, minimally invasive surgeries for local tumor treatment have been developed in addition to invasive surgeries such as laparotomy. Non-invasive surgeries have also been developed using a gamma knife, a cyber knife, or a high intensity focused ultrasound (HIFU) knife, for example, and these tools have become commercially available. In particular, recently commercialized HIFU knives, which use ultrasound, are widely used for non-harmful and environmentally friendly treatment.

Treatments using a HIFU knife include surgeries for removal and treatment of tumors, in which HIFU is focused on a tumor site to be treated and irradiated thereinto, thereby causing local destruction or necrosis of tumor tissues.

### SUMMARY

Provided are systems for diagnosing and treating a tumor by real-time monitoring of the necrosis of a tumor using ultrasound.

Provided are computer readable recording media that record a program to be executed in a computer.

Additional aspects will be set forth in part in the description which follows and, in part, will be apparent from the description, or may be learned by practice of the presented embodiments.

According to an aspect, a method of treatment and diagnosis using ultrasound includes inducing a shear wave around a treatment site into which ultrasound for treatment is irradiated, by using the ultrasound for treatment; irradiating ultrasound for diagnosis into the treatment site; obtaining a degree of a change in the properties of a tissue of the treatment site by using a displacement of the shear wave measured by an echo ultrasound produced such that the ultrasound for diagnosis is reflected; and determining a degree of treatment of the tissue of the treatment site based on the obtained change in the properties of the tissue.

According to another aspect, there is provided a computer readable recording medium recording a program for executing the method of treatment and diagnosis using ultrasound on a computer.

According to another aspect, a system for treatment and diagnosis using ultrasound includes an ultrasonic treatment device to irradiate ultrasound for treatment into a tumor site that is to be treated and induce a shear wave around the tumor site by using the ultrasound for treatment; an ultrasonic diagnosis device to irradiate ultrasound for diagnosis into the tumor site and receive an echo ultrasound produced such that the ultrasound for diagnosis is reflected; and a processor to obtain a degree of a change in the properties of a tissue of the tumor site by using a displacement of the shear wave measured by the echo ultrasound and determining a degree of treatment of the tissue of the tumor site based on the degree of a change in the properties of the tissue.

### BRIEF DESCRIPTION OF THE DRAWINGS

The patent or application file contains at least one drawing executed in color. Copies of this patent or patent application publication with color drawings will be provided by the Office upon request and payment of the necessary fee. These and/or other aspects will become apparent and more readily appreciated from the following description of the embodiments, taken in conjunction with the accompanying drawings of which:

FIG. 1 is a diagram of a system for treatment and diagnosis using ultrasound, according to an embodiment;

FIG. 2 is a diagram of a processor, according to an embodiment;

FIG. 3A is a view for explaining a shear wave, according to an embodiment;

FIG. 3B illustrates images of a shear wave induced around a treatment site, according to an embodiment;

FIG. 3C illustrates images showing a shear wave induced in a multifocal region, according to an embodiment;

FIG. 4 is a diagram for explaining a method of irradiating a defocusing-type plane wave in an ultrasonic diagnosis device, according to an embodiment;

FIG. 5A illustrates images and graphs showing a defocusing-type plane wave, according to an embodiment;

FIG. 5B illustrates images and graphs showing a general plane wave;

FIG. 6 is a diagram illustrating a process of generating ultrasound images with regards to displacement of a shear wave, according to an embodiment;

FIG. 7 is a diagram for explaining a process of measuring the displacement of a shear wave and a process of calculating the shear modulus of a tissue, according to an embodiment;

FIG. 8 illustrates graphs showing a degree of treatment of a tissue, according to an embodiment;

FIGS. 9A and 9B respectively illustrate a graph and a table, each of which shows results of comparison of a case where a method of inducing a shear wave using ultrasound for treatment according to an embodiment is used with a case where a method of inducing a shear wave using general ultrasound for diagnosis is used;

FIG. 10 is a flowchart illustrating a method for treatment and diagnosis using ultrasound, according to an embodiment;

FIG. 11 is a flowchart particularly illustrating an operation of obtaining a degree of a change in the properties of a tissue (operation 1103) of FIG. 10, according to an embodiment;

FIG. 12 is a flowchart illustrating a method for treatment and diagnosis using ultrasound on all tumor sites, according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

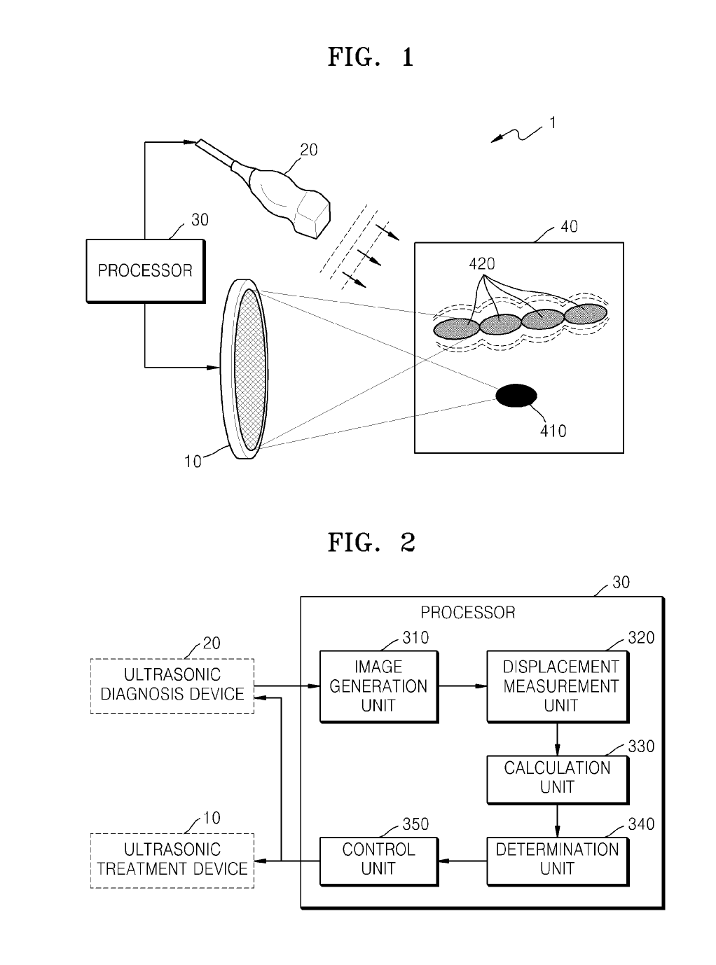

FIG. 1 is a diagram of a system 1 for treatment and diagnosis using ultrasound, according to an embodiment. Referring to FIG. 1, the system 1 for treatment and diagnosis using ultrasound includes an ultrasonic treatment device 10, an ultrasonic diagnosis device 20, and a processor 30.

In FIG. 1, only the elements relevant to the present embodiment are illustrated. Thus, it will be understood by one of ordinary skill in the art that the system 1 for treatment and diagnosis using ultrasound may further include other commonly used elements, in addition to the elements illustrated in FIG. 1.

The system 1 for treatment and diagnosis using ultrasound treats a tumor site 40 of a patient by using ultrasound for treatment and monitors the results of treatment by using ultrasound for diagnosis.

In particular, in the system 1 for treatment and diagnosis using ultrasound, when a tumor develops in the body of a patient, the ultrasonic treatment device 10 irradiates ultrasound for treatment into the tumor site 40, thereby generating a lesion, and the ultrasonic diagnosis device 20 obtains ultrasound images of the tumor site 40, thereby diagnosing whether or not the treatment is completed. Such a lesion is obtained as a result of local destruction or necrosis of a tissue of the tumor site 40. That is, the system 1 for treatment and diagnosis using ultrasound is a medical system for diagnosing a patient by causing the necrosis of a tumor by ultrasonic treatment and monitoring the results of treatment.

In particular, the ultrasound for treatment may be high intensity focused ultrasound (HIFU). Thus, the ultrasonic treatment device 10 may be a device that irradiates the HIFU as ultrasound for treatment. For convenience of explanation, unless otherwise stated herein, the ultrasound for treatment is the HIFU. However, it will be understood by one of ordinary skill in the art that the ultrasound for treatment is not limited to the above example, and may be any other focused ultrasound similar to the HIFU.

The operation of each element of the system 1 for treatment and diagnosis using ultrasound will now be described.

The ultrasonic treatment device 10 irradiates the ultrasound for treatment into a tumor site to be treated 410 (hereinafter, referred to as “treatment site 410”), and induces a shear wave around the treatment site 410 by using the ultrasound for treatment. In this regard, the treatment site 410 is a local site of the tumor site 40 to which the ultrasound for treatment is intensively irradiated, according to a tumor treatment plan using ultrasound for treatment.

The ultrasonic treatment device 10 induces a shear wave in each of multifocal regions generated around the treatment site 410 by using the ultrasound for treatment. Around the treatment site 410 corresponds to a left side region (the position of focal points 420 in FIG. 1) or a right side region (not shown) of the treatment site 410 with respect to a direction in which the ultrasound for treatment proceeds. The ultrasonic treatment device 10 may irradiate the ultrasound for treatment in such a manner that the treatment site 410 and the multifocal regions (corresponding to the position of focal points 420 in FIG. 1) in the left side region where the shear wave is induced do not overlap each other. This is because the shear wave generated in the multifocal regions is generated weakly in a central axis of the focal points 420 and generated strongly around the central axis thereof. Thus, to strongly induce the shear wave around the treatment site 410 to be observed, the treatment site 410 may not overlap with the multifocal regions.

To induce the shear wave, ultrasound for diagnosis is generally used. However, the ultrasonic diagnosis device 20 provides a low output and includes a small sized transducer. In addition, the ultrasound for diagnosis has a high frequency. Thus, the shear wave is generally induced only to approximately 4 cm in depth from the skin. If the shear wave is induced more deeply into the skin, a very high output is required, and thus the deeper induction of the shear wave into the skin is inefficient.

On the other hand, an ultrasonic transducer for treatment using HIFU has a low frequency, and thus ultrasound is barely attenuated. In addition, the ultrasonic transducer for treatment has a large aperture, and thus, when the ultrasound is focused, it is capable of concentrating high intensity energy on a local site. Thus, the ultrasonic transducer for treatment may induce a shear wave more deeply into the tissue at a relatively low output. Accordingly, a HIFU transducer for treatment of the ultrasonic treatment device 10 is used in the induction of the shear wave, together with the treatment of the treatment site 410.

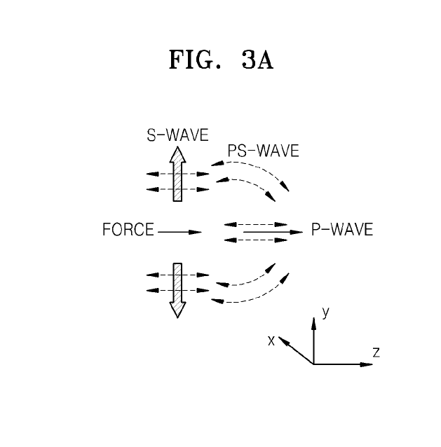

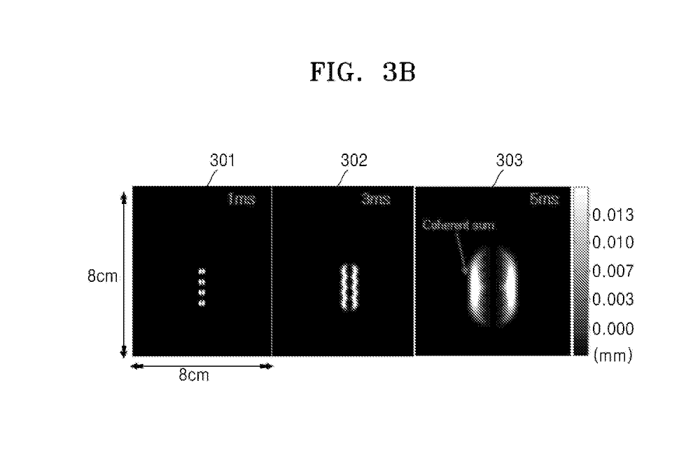

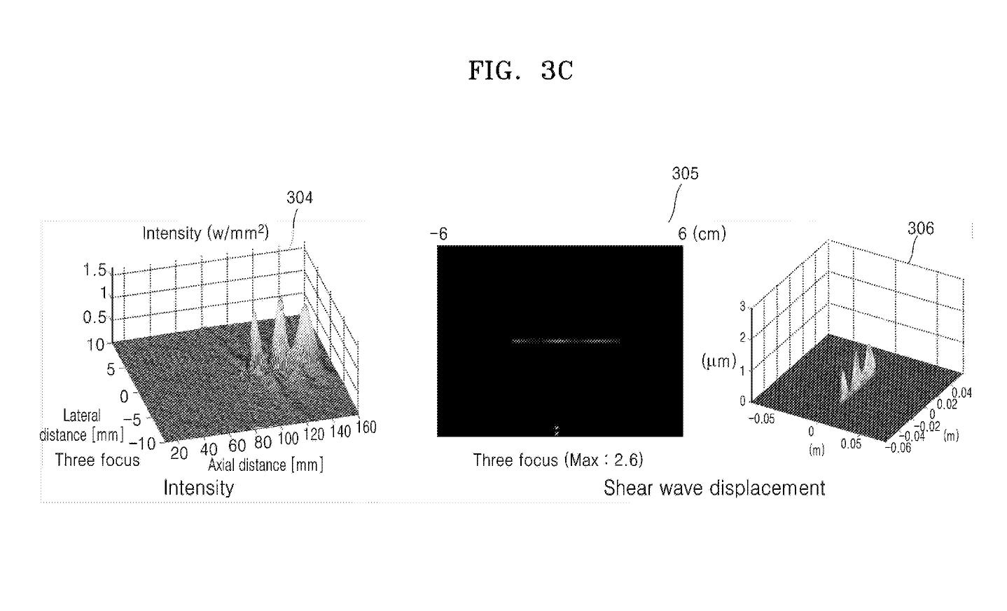

FIG. 3A is a view for explaining a shear wave, according to an embodiment. Referring to FIG. 3A, when a force of a point impulse is applied in a Z-axis direction, a longitudinal wave (P-wave), a transverse wave (S-wave), and a wave coupled with the P-wave and the S-wave (PS-wave) are generated. In this regard, a shear wave, S-wave, vibrates in a direction in which a wave proceeds from a vibration source to which the force of the point impulse is applied, and proceeds in a Y-axis direction.

The displacement in the Z-axis direction of the shear wave, S-wave, is represented by Equation 1 below:

$${g}_{zz}^{s} ( r , t ) = \frac{1}{4 π ρ {c}_{s}} \frac{1}{\sqrt{2 π {v}_{s} t}} \frac{{r}^{2} - {z}^{2}}{{r}^{2}} {e}^{- \frac{{( t - r / {c}_{s} )}^{2} {c}_{s}^{2}}{2 {v}_{s} t}} \qquad Equation 1$$

wherein g_{zz}^{s} refers to the displacement in the Z-axis direction of the S-wave, ρ refers to density, cₛ refers to the velocity of shear wave, vₛ refers to viscosity, and r is a distance from the origin.

FIG. 3B illustrates images of a shear wave induced around a site to be treated, according to an embodiment. In FIG. 3B, images 301, 302, and 303 illustrate a shear wave intensity respectively obtained 1 ms, 3 ms, and 5 ms after the shear wave was induced.

Referring to FIG. 3B, it is seen that the shear wave was induced in each of 4 multifocal regions (corresponding to the position of focal points 420 in FIG. 1). The ultrasonic treatment device 10 induces the shear wave in each of the four multifocal regions using the HIFU. In this regard, the ultrasonic treatment device 10 nearly simultaneously induces the shear wave in each multifocal region. The number of multifocal regions is not limited to the above example, and may be variously selected.

Referring to the image 301, the shear wave is induced in each multifocal region, and barely proceeds in a direction of the Y-axis. In comparison, referring to the image 302, the shear wave proceeds a little further in each multifocal region in a direction of the Y-axis.

Referring to the image 303, the shear wave induced in each multifocal region coheres with each other, and, as a result, a coherent sum of the shear waves is generated. Therefore, when the shear wave is induced in each multifocal region, a more powerful shear wave may be induced by the coherent sum of the shear waves, as compared to a case where a shear wave is induced in a focal region.

As described above, the shear wave is induced in the multifocal regions (corresponding to the position of the focal points 420 in FIG. 1) around the treatment site 410, and the multifocal regions correspond to a side region of the treatment site 410 with respect to a direction in which the ultrasound for treatment proceeds. Since the shear wave is induced in the side region of the treatment site 410, a powerful shear wave may be transmitted to the treatment site 410 by the coherent sum of the shear waves, as time passes. Thus, the displacement of the shear wave may be measured more distinctly in the treatment site 410 due to the transmittance of the powerful shear wave. That is, when the shear wave is induced in the multifocal regions, the displacement of the shear wave may be measured more distinctly at a broader range, as compared to a case where a shear wave is induced in a focal region.

FIG. 3C illustrates images showing a shear wave induced in multifocal regions, according to an embodiment. Referring to FIG. 3C, image 304 three-dimensionally illustrates the distribution of the intensity of the shear wave evaluated at an aspect of a Z-axial distance and a lateral distance. In this regard, the shear wave is induced using ultrasound for treatment. Referring to image 304, it is seen that a relatively high intensity of a shear wave is induced at a distance between approximately 100 mm to approximately 140 mm. In addition, in FIG. 3C, image 306 three-dimensionally illustrates the displacement of the shear wave induced in each of three multifocal regions.

Referring back to FIG. 1, the ultrasonic diagnosis device 20 may also be referred to as a probe for diagnosis, and irradiates ultrasound for diagnosis into the treatment site 410 and receives an echo ultrasound produced such that the irradiated ultrasound for diagnosis is reflected. In this regard, the ultrasonic diagnosis device 20 irradiates the ultrasound for diagnosis into the treatment site 410 and receives the echo ultrasound, in order to generate ultrasound images for diagnosis of the treatment site 410. A general process of generating the ultrasound images by using the echo ultrasound will be obvious to one of ordinary skill in the art, and thus a detailed description thereof is not provided herein.

The ultrasonic diagnosis device 20 irradiates a defocusing-type plane wave as the ultrasound for diagnosis.

The reason why the defocusing-type plane wave is used as the ultrasound for diagnosis is that the shear wave may be observed at a broader range and the displacement of the shear wave may be measured more accurately. In particular, since the ultrasonic diagnosis device 20 uses the defocusing-type ultrasound for diagnosis, the displacement of the shear wave may be measured at a broader range in the case where a focusing-type ultrasound for diagnosis is used. In addition, by using the plane wave of which intensity is maintained relatively constant even though the plane wave reaches deeply into the body of a human, the displacement of the shear wave may be measured more accurately than the case where a spherical wave whose intensity becomes weaker as the spherical wave reaches more deeply into the body of a human is used.

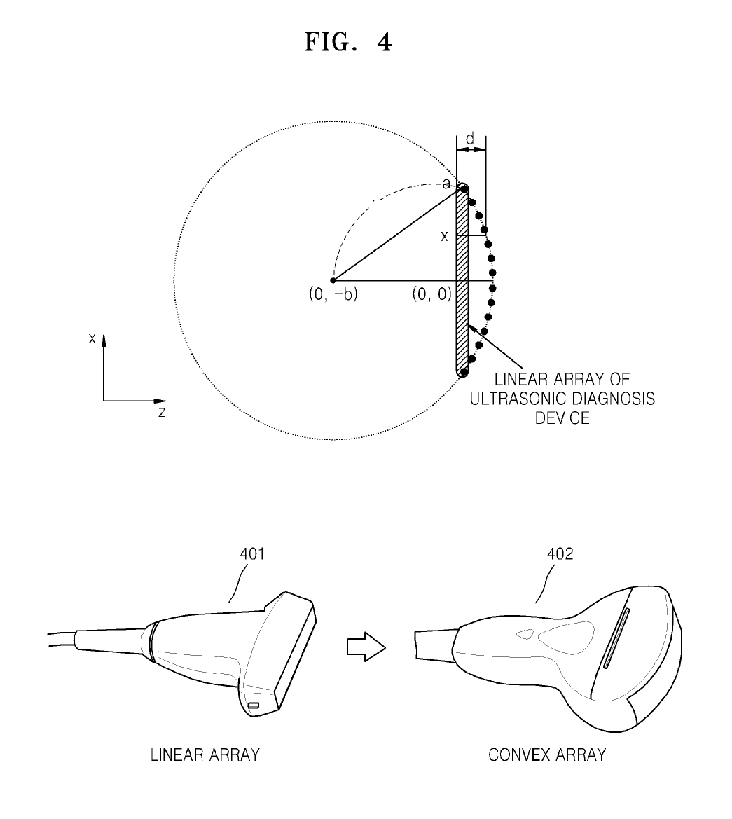

The ultrasonic diagnosis device 20 may use convex array-type transducer elements and linear array-type transducer elements, and irradiate the defocusing-type plane wave. A detailed description thereof will now be provided with reference to FIG. 4.

FIG. 4 is a diagram for explaining a method of irradiating a defocusing-type plane wave in an ultrasonic diagnosis device 20, according to an embodiment. Referring to FIG. 4, the ultrasonic diagnosis device 20 includes linear array-type transducer elements.

The ultrasonic diagnosis device 20 irradiates, with respect to a direction of the Z-axis, which is a direction in which the ultrasound for diagnosis proceeds, ultrasound for diagnosis in such a manner that the irradiation of the ultrasound for diagnosis becomes delayed towards sides of an array of the ultrasonic diagnosis device 20.

In detail, assuming a linear array is a chord of a circle with a radius r, in order for the ultrasound for diagnosis to be irradiated in the form of an arc corresponding to the chord, the ultrasonic diagnosis device 20 adjusts the irradiation timing of each transducer elements of the linear array. The irradiation timing of each transducer element of the linear array is obtained using Equation 2 below:

$$b = \sqrt{{r}^{2} - {a}^{2}} \\ d = r ( 1 - \frac{b}{{x}^{2} + {b}^{2}} ) \\ delay ( time ) = \frac{d}{c ( velocity )} . \qquad Equation 2$$

Therefore, even when the ultrasonic diagnosis device 20 includes linear array-type transducer elements, the irradiation timing of the transducer elements is adjusted as described above, and thus a linear array-type diagnosis probe 401 may be operated like a convex array-type diagnosis probe 402 with a radius r. That is, the irradiation timing of the transducer elements of the linear array-type diagnosis probe 401 is adjusted as described above, and thus the defocusing-type plane wave may be irradiated from the linear array-type diagnosis probe 401.

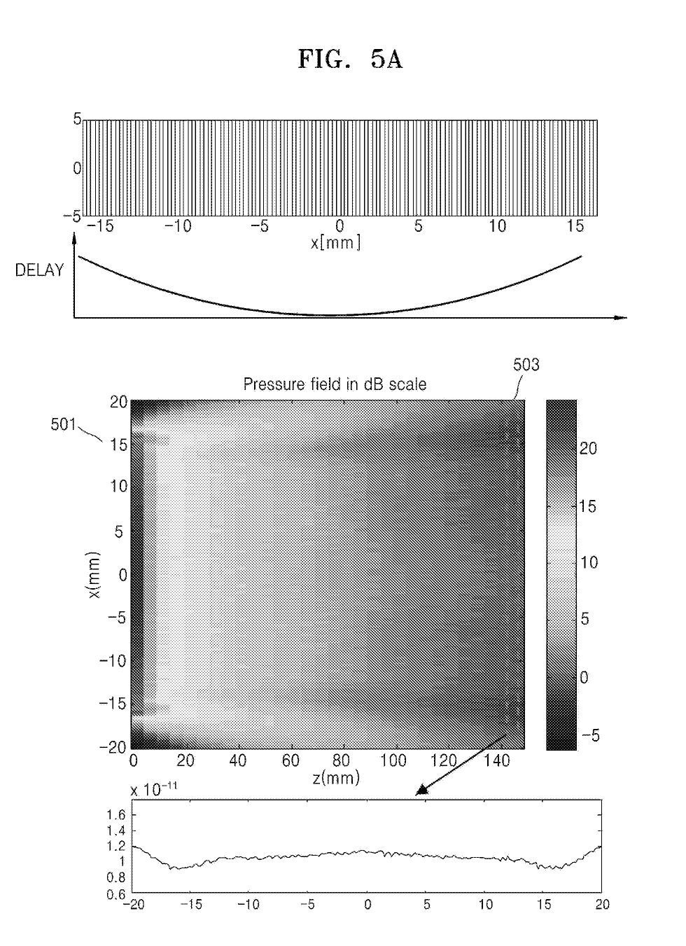

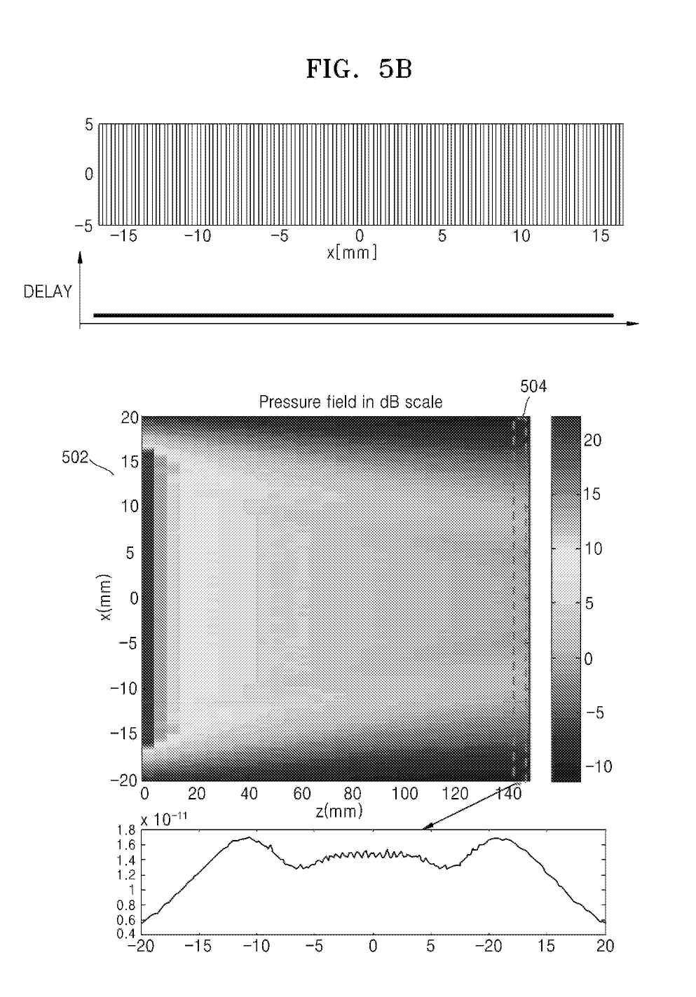

FIG. 5A illustrates images and graphs showing a defocusing-type plane wave 501, according to an embodiment. FIG. 5B illustrates images and graphs showing a general plane wave 502. Referring to FIGS. 5A and 5B, it is seen that a range of the wave transmitted at a point 503 of approximately 140 mm in the body of a human when the defocusing-type plane wave 501 is irradiated is wider than the wave transmitted at a point 504 of approximately 140 mm in the body of a human when the general plane wave 502 is irradiated. Therefore, the shear wave may be observed at a broader range than the case where a general plane wave is used, by using the ultrasonic diagnosis device 20 that uses the defocusing-type plane wave 501.

The shear wave is used to obtain a degree of a change in the properties of a tissue. In particular, the elasticity of a tissue in the body of a human is changed due to tissue necrosis. That is, necrosis of the tissue causes the tissue to harden, resulting in an increase in the elasticity of the tissue. Therefore, whether or not tissue necrosis occurs may be determined using the properties such as a change in the elasticity of the tissue. In particular, shear modulus is used to measure a change in elasticity by value. The change in elasticity of the treatment site 410 may be determined by measuring the displacement of shear wave in the treatment site 410, which will be described later in more detail.

Unless otherwise stated herein, a change in the properties of a tissue corresponds to a change of the shear modulus of a tissue. However, the change in the properties of a tissue is not limited to the above example, and may include a change in the viscosity of a tissue.

Referring back to FIG. 1, the processor 30 obtains a degree of a change in the properties of a tissue of the treatment site 410 by using the displacement of the shear wave measured by the echo ultrasound, and determines whether necrosis of the tissue of the treatment site 410 occurs based on the degree of a change in the properties of the tissue.

FIG. 2 is a diagram of a processor 30, according to an embodiment. Referring to FIG. 2, the processor 30 includes an image generation unit 310, a displacement measurement unit 320, a calculation unit 330, a determination unit 340, and a control unit 350. The processor 30 may be configured as an array of a plurality of logic gates, or may be configured as a combination of a commonly used microprocessor and a memory in which a program executable on the microprocessor is stored. In addition, it will be understood by one of ordinary skill in the art that the processor 30 may be configured using other types of hardware.

The control unit 350 controls an operation of the ultrasonic treatment device 10 and the ultrasonic diagnosis device 20. For example, the control unit 350 determines to which portion of the treatment site 410 the ultrasonic treatment device 10 irradiates ultrasound for treatment, at what intensity the ultrasound for treatment is irradiated, and in which position of multifocal regions the ultrasonic treatment device 10 induces a shear wave, and then controls the operations of the ultrasonic treatment device 10. In addition, the control unit 350 controls the irradiation timing of each of the transducer elements of the ultrasonic diagnosis device 20. Furthermore, it will be understood by one of ordinary skill in the art that the control unit 350 may further control general operations of the ultrasonic treatment device 10 and the ultrasonic diagnosis device 20.

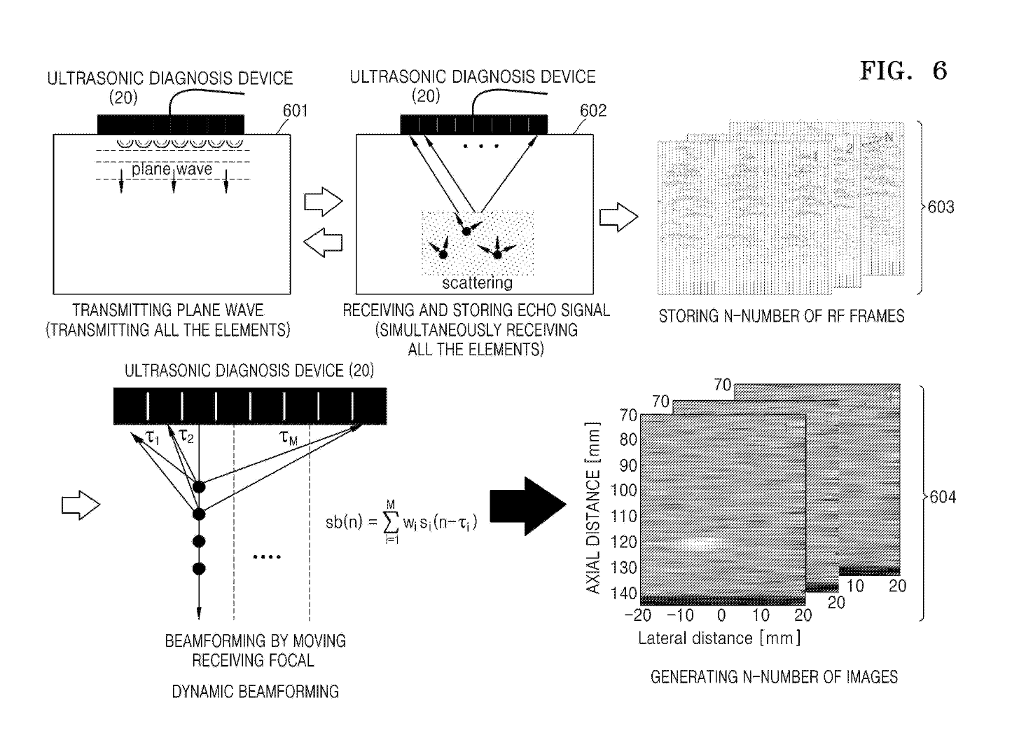

The image generation unit 310 generates ultrasound images of the treatment site 410 by using the echo ultrasound. As described above, a general process of generating ultrasound images by using the echo ultrasound will be obvious to one of ordinary skill in the art, and thus a detailed description thereof is not provided herein. The process of generating ultrasound images will now be described with reference to FIG. 6.

FIG. 6 is a diagram illustrating a process of generating ultrasound images with regards to the displacement of a shear wave, according to an embodiment.

Referring to FIG. 6, the ultrasonic diagnosis device 20 irradiates a defocusing-type plane wave, such as quasi-plane wave, as ultrasound for diagnosis (Operation 601).

The ultrasonic diagnosis device 20 receives an echo ultrasound that is scattered and reflected from a tissue of the body of a human (Operation 602).

A storage unit (not shown) converts a signal of the echo ultrasound into a digital signal and then stores the digital signal as N (where N is a natural number) radio frequency (RF) frames (Operation 603). A shear wave proceeds at the rate of 1 to 10 m/s in a tissue of the body of a human. Thus, to observe the shear wave at a resolution of several millimeters, ultrasound images should be obtained in a unit of a thousand frames per second. In this regard, to rapidly observe the shear wave by obtaining a thousand frames per second of ultrasound images, a defocusing-type (or unfocusing-type) plane wave is required as the ultrasound for diagnosis.

The image generation unit 310 performs beam forming by using the stored N-number of RF frames, thereby generating N two-dimensional ultrasound images (Operation 604).

Referring back to FIG. 2, the displacement measurement unit 320 measures the displacement of the shear wave by cross-correlating the generated ultrasound images.

The calculation unit 330 calculates the shear modulus of a tissue of the treatment site 410 by using the measured displacement of the shear wave.

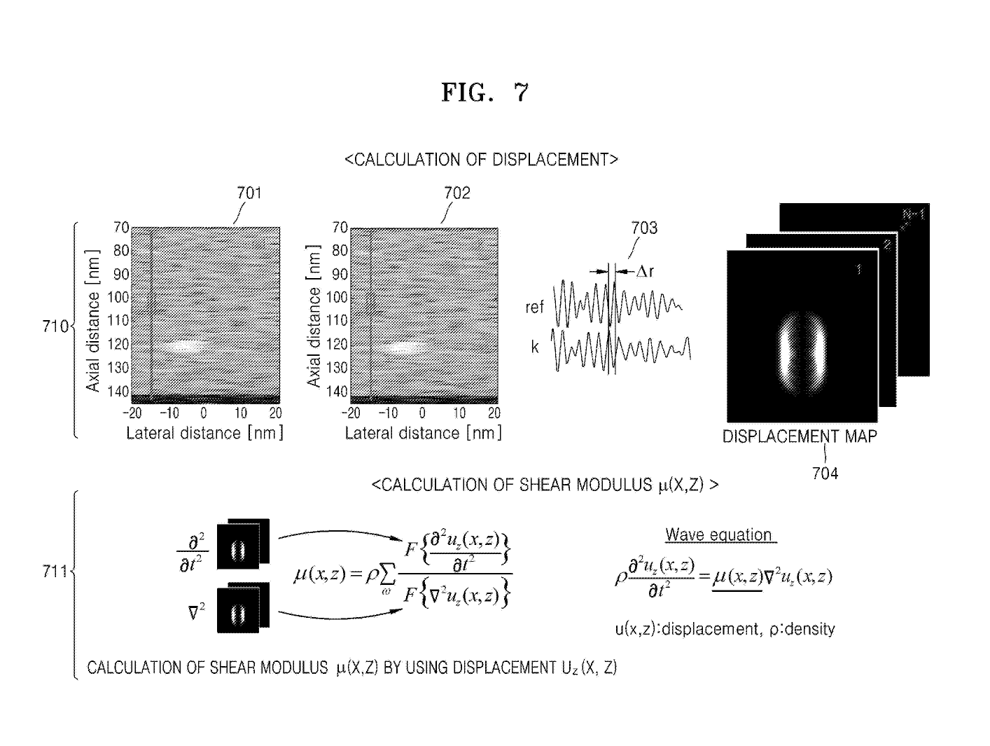

FIG. 7 is a diagram for explaining a process of measuring the displacement of a shear wave and a process of calculating the shear modulus of a tissue, according to an embodiment. Referring to FIG. 7, a process of measuring the displacement of a shear wave (Operation 710) and a process of calculating the shear modulus of a tissue (Operation 711) are illustrated.

In operation 710, first, the displacement measurement unit 320 cross-correlates two ultrasound images 701 and 702 adjacent to each other on a time basis (Operation 703). Through operation 703, the displacement measurement unit 320 measures a movement distance Δr of the shear wave between the two ultrasound images 701 and 702. Δr corresponds to r of Equation 1 described above, and thus the displacement measurement unit 320 calculates the displacement u_{z}(x,z) of the shear wave by using Equation 1. In this regard, the displacement u_{z}(x,z) of the shear wave corresponds to g_{zz}^{s} of Equation 1. When the displacement measurement unit 320 calculates all the time-based displacements of the shear wave by using each ultrasound image, the displacement measurement unit 320 produces time-based displacement maps 704 of the shear wave by using the displacements of the shear wave measured on a time basis.

Operation 711 will now be described in detail. A known wave equation is represented by Equation 3 below:

$$ρ \frac{{\partial }^{2} {u}_{z} ( x , z )}{\partial {t}^{2}} = μ ( x , z ) {∇}^{2} {u}_{z} ( x , z ) \qquad Equation 3$$

wherein u_{z}(x,z) indicates the displacement of a shear wave, obtained by Equation 1, and ρ refers to density.

Correlations among shear modulus μ, density ρ and velocity v are represented by Equation 4 below:

$$v = \sqrt{\frac{μ}{ρ}} ⇒ μ = ρ {v}^{2} . \qquad Equation 4$$

Equation 5, which is used to calculate the shear modulus, is induced from Equation 3 and Equation 4:

$$μ ( x , z ) = ρ {\sum }_{ω} \frac{F \{ \frac{{\partial }^{2} {u}_{z} ( x , z )}{\partial {t}^{2}} \}}{F \{ {∇}^{2} {u}_{z} ( x , z ) \}} . \qquad Equation 5$$

Referring to Equation 5, the displacement of the shear wave is subjected to time-resolved Fourier transformation and space-resolved Fourier transformation based on the time-based displacement maps 704 of the shear wave, thereby obtaining a term relevant to v² of Equation 4. Thus, the shear modulus μ(x,z) may be obtained by Equation 5 by using the displacement u_{z}(x,z) of the shear wave.

Referring back to FIG. 2, the determination unit 340 determines whether tissue necrosis occurs based on a change in the properties of the tissue corresponding to a change in the calculated shear modulus. In particular, the determination unit 340 determines based on the change in the shear modulus of the tissue that the tissue necrosis occurs at a time when an inflection point, where the elasticity of the tissue increases as time spent treating the treatment site 410 by using the ultrasound for treatment passes, appears.

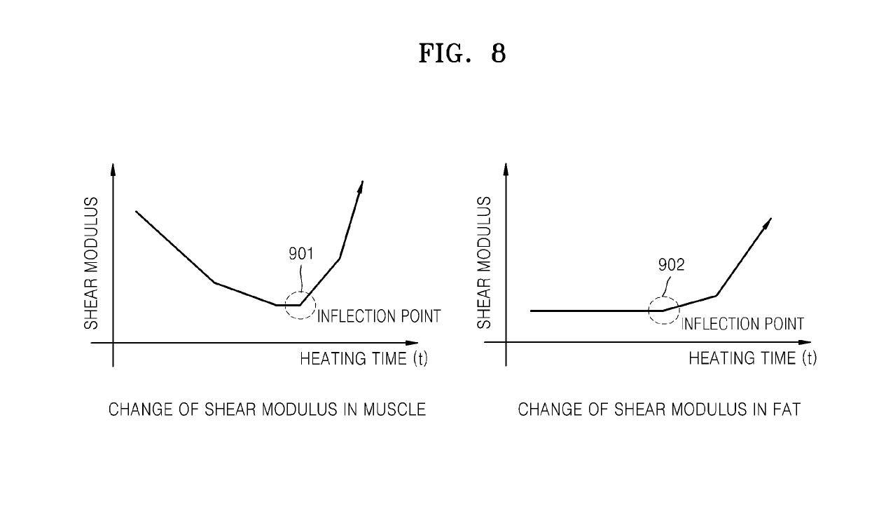

FIG. 8 illustrates graphs showing the degree of treatment of a tissue, according to an embodiment. In FIG. 8, a graph showing a change in the shear modulus in muscle and a graph showing a change in the shear modulus in fat are illustrated. As described above, the determination unit 340 may determine based on a change in the shear modulus of a tissue that the tissue necrosis begins or is completely terminated at a time when an inflection point 901 or 902, where the elasticity of the tissue increases as time spent treating the treatment site 410 by using the ultrasound for treatment passes, appears.

Until the determination unit 340 determines that the necrosis of the tissue of the treatment site 410 is completed, the ultrasonic treatment device 10 repeatedly irradiates the ultrasound for treatment into the treatment site 410 and the ultrasonic diagnosis device 20 repeatedly induces a shear wave, thereby obtaining a degree of a change in the properties of the tissue of the treatment site 410.

In detail, in an ultrasonic treatment process using ultrasound for treatment such as HIFU, when the HIFU is irradiated into the treatment site 410, the temperature of the tumor site to be treated momentarily increases, and thus a tissue and a blood vessel of the treatment site 410 are subjected to coagulative necrosis.

In the present embodiment, only the treatment site 410 is illustrated as a site to be treated. That is, until it is determined that the necrosis of the tissue of the treatment site 410 occurs, the ultrasonic treatment device 10 repeatedly irradiates the ultrasound for treatment into the treatment site 410 and the ultrasonic diagnosis device 20 repeatedly induces a shear wave, thereby obtaining a degree of a change in the properties of the tissue of the treatment site 410.

However, such a process is also repeatedly performed on other sites to be treated according to a treatment plan, and is performed until the treatment (or necrosis) of all the treatment sites of the tumor site 40 is completed.

That is, when it is determined that the treatment of the treatment site 410 is completed due to the necrosis of the tissue of the treatment site 410, the control unit 350 controls the ultrasonic treatment device 10 and the ultrasonic diagnosis device 20 to operate on other treatment sites according to a treatment plan.

The determination unit 340 may further determine that the tumor site 40 is completely treated according to a treatment plan. In this case, the control unit 350 terminates the treatment and diagnosis using the ultrasonic treatment device 10 and the ultrasonic diagnosis device 20.

The system 1 for treatment and diagnosis using ultrasound continually monitors whether the necrosis of the tissue of the treatment site 410 occurs, thereby treating the tumor site 40 by using ultrasound for treatment.

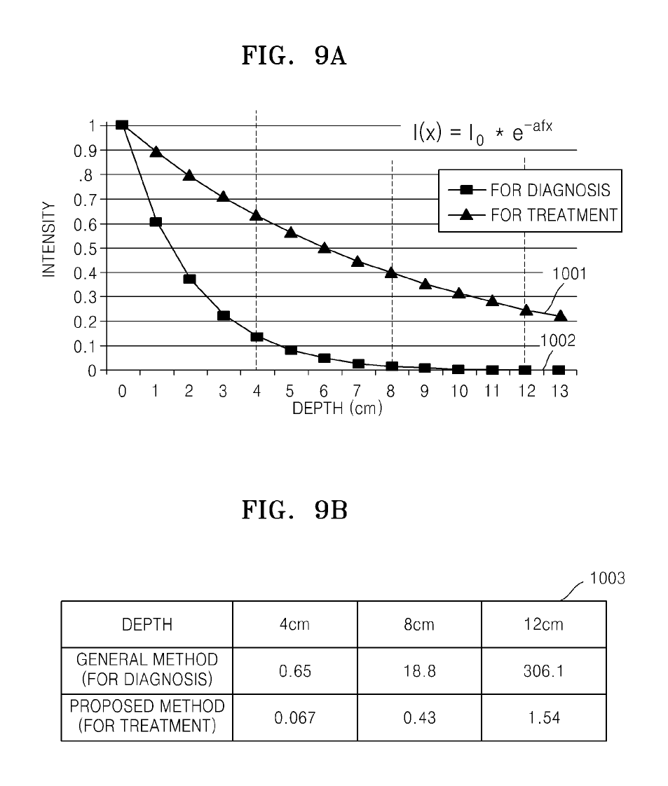

FIGS. 9A and 9B respectively illustrate a graph and a table, each of which shows the results of comparison of a case where a method of inducing a shear wave using ultrasound for treatment according to an embodiment is used with a case where a method of inducing a shear wave using general ultrasound for diagnosis is used. The present embodiment is only an experimental example for convenience of explanation.

In FIG. 9A, a graph showing the attenuation of the intensity of a shear wave in the case where the shear wave is induced using ultrasound for treatment (curve 1001) and in the case where the shear wave is induced using general ultrasound for diagnosis (curve 1002) is illustrated. When the ultrasound for diagnosis is used, the intensity of the shear wave sharply decreases to a depth of approximately 4 cm and is nearly zero from a depth of approximately 8 cm or greater. On the other hand, when the ultrasound for treatment is used, the intensity of the shear wave slowly decreases to a depth of approximately 12 cm, and thus the induction of the shear wave is more effective than when the ultrasound for diagnosis is used.

FIG. 9B illustrates a table showing a surface output of a transducer required for inducing the displacement of a constant shear wave. In particular, table 1003 of FIG. 9B shows the results of comparison of the case where ultrasound for diagnosis is used (general method) with the case where ultrasound for treatment according to an embodiment is used. In this regard, the displacement of a constant shear wave is 28 μm. In the general method, it is known that the application of a pressure of 40 bar (based on water) at a focal depth of 4 cm is required to induce the displacement of the shear wave by 28 μm. Thus, the comparison is conducted on that basis. A probe of the general ultrasound for diagnosis was assumed to have a width of 4 cm and a frequency of 4.3 MHz, and the transducer of the ultrasonic treatment device 10 was assumed to have a diameter of 12 cm and a frequency of 1 MHz. 0.5 dB/MHz/cm was used as an attenuation coefficient.

As illustrated in FIG. 9B, to induce the displacement of the shear wave by 28 μm at a focal depth of 4 cm, an output of 0.65 W is required in the general ultrasound for diagnosis while an output of 0.067 W is required in the ultrasound for treatment. In contrast, at a focal depth of 12 cm, an output of 306.1 W is required in the general ultrasound for diagnosis while the ultrasound for treatment only requires a much lower output, i.e., 1.54 W. Thus, the induction of the shear wave in a deep tissue is more effectively performed by using the ultrasound for treatment than when the general ultrasound for diagnosis is used.

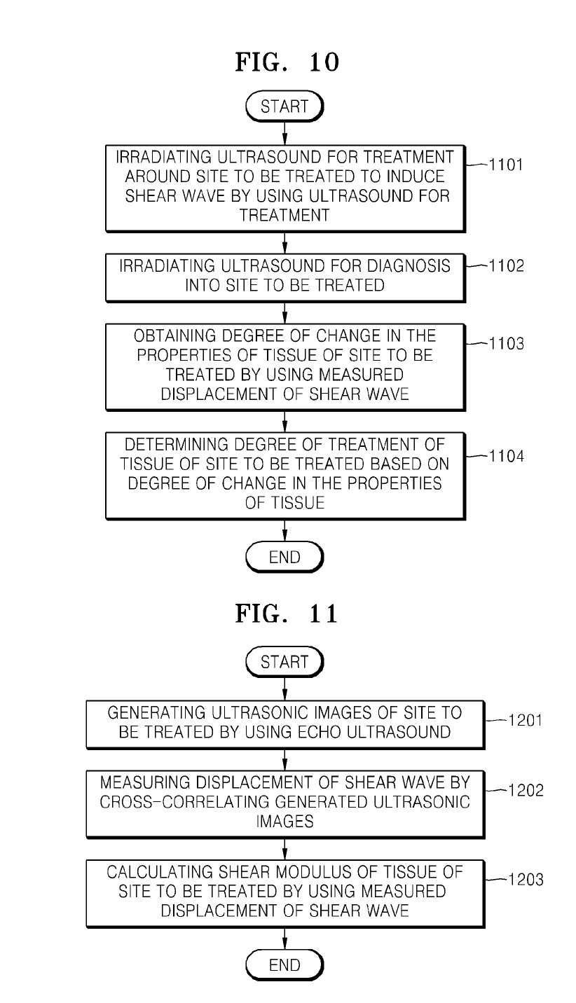

FIG. 10 is a flowchart illustrating a method for treatment and diagnosis using ultrasound, according to an embodiment. FIG. 10 illustrates time-series operations performed in the system 1 for treatment and diagnosis using ultrasound illustrated in FIGS. 1 and 2. A detailed description of the system 1 for treatment and diagnosis using ultrasound illustrated in FIGS. 1 and 2 has already been provided above, and thus it will not be provided in the present embodiment.

The ultrasonic treatment device 10 irradiates ultrasound for treatment into the treatment site 410, and induces a shear wave around the treatment site 410 (corresponding to the position of focal points 420 in FIG. 1) by using the ultrasound for treatment (Operation 1101).

The ultrasonic diagnosis device 20 irradiates ultrasound for diagnosis into the treatment site 410, and receives an echo ultrasound produced such that the ultrasound for diagnosis is reflected (Operation 1102).

The processor 30 obtains a degree of a change in the properties of a tissue of the treatment site 410 by using the displacement of the shear wave measured by the echo ultrasound (Operation 1103).

The processor 30 determines the degree of treatment of the tissue of the treatment site 410, based on the degree of a change in the properties of the tissue thereof (Operation 1104).

FIG. 11 is a flowchart particularly illustrating an operation of obtaining a degree of a change in the properties of a tissue (operation 1103) of FIG. 10, according to an embodiment.

The image generation unit 310 generates ultrasonic images of the tumor site to be treated by using the echo ultrasound (Operation 1201).

The displacement measurement unit 320 measures the displacement of the shear wave by cross-correlating the generated ultrasonic images (Operation 1202).

The calculation unit 330 calculates the shear modulus of the tissue of the treatment site 410 by using the measured displacement of the shear wave (Operation 1203).

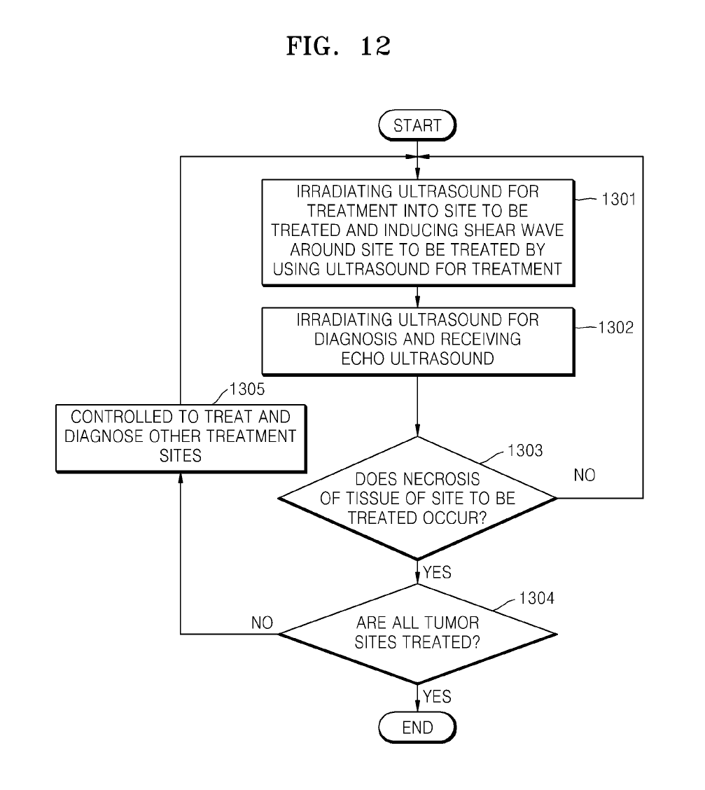

FIG. 12 is a flowchart illustrating a method for treatment and diagnosis using ultrasound on all tumor sites, according to an embodiment. Comparing FIG. 11 with FIG. 12, FIG. 11 is a flowchart illustrating a method for treatment and diagnosis on the treatment site 410 while FIG. 12 is a flowchart illustrating a method for treatment and diagnosis on the tumor site 40 including the treatment site 410 and other tumor sites to be treated.

The ultrasonic treatment device 10 irradiates ultrasound for treatment into a treatment site, and induces a shear wave around the treatment site by using the ultrasound for treatment (Operation 1301).

The ultrasonic diagnosis device 20 irradiates ultrasound for diagnosis into the treatment site, and receives an echo ultrasound produced such that the ultrasound for diagnosis is reflected (Operation 1302).

The processor 30 obtains a degree of a change in the properties of a tissue of the treatment site by using the displacement of the shear wave measured by the echo ultrasound, and determines whether necrosis of the tissue occurs, based on the degree of a change in the properties of the tissue (Operation 1303).

The processor 30 determines whether all tumor sites are treated according to a treatment plan (Operation 1304).

The processor 30 controls the ultrasonic treatment device 10 and the ultrasonic diagnosis device 20 to treat and diagnose other treatment sites according to the treatment plan (Operation 1305). That is, until all of the tumor sites are treated, operations 1301 through 1303 are repeatedly performed on each of the other treatment sites.

The above-described embodiments may be recorded in computer-readable media including program instructions to implement various operations embodied by a computer. The media may also include, alone or in combination with the program instructions, data files, data structures, and the like. The program instructions recorded on the media may be those specially designed and constructed for the purposes of embodiments, or they may be of the kind well-known and available to those having skill in the computer software arts. Examples of computer-readable media include magnetic media such as hard disks, floppy disks, and magnetic tape; optical media such as CD ROM disks and DVDs; magneto-optical media such as optical disks; and hardware devices that are specially configured to store and perform program instructions, such as read-only memory (ROM), random access memory (RAM), flash memory, and the like. The computer-readable media may also be a distributed network, so that the program instructions are stored and executed in a distributed fashion. The program instructions may be executed by one or more processors. The computer-readable media may also be embodied in at least one application specific integrated circuit (ASIC) or Field Programmable Gate Array (FPGA), which executes (processes like a processor) program instructions. Examples of program instructions include both machine code, such as produced by a compiler, and files containing higher level code that may be executed by the computer using an interpreter. The above-described devices may be configured to act as one or more software modules in order to perform the operations of the above-described embodiments, or vice versa.

As described above, according to the one or more of the above embodiments, a more powerful shear wave may be induced in a tissue of a tumor at a relatively low output by using ultrasound for treatment, as compared to when ultrasound for diagnosis is used. In addition, when a shear wave is induced in multifocal regions by using ultrasound for treatment, a more powerful shear wave may be induced in a tumor site to be treated and also be induced in a wider area, as compared to when a shear wave is induced in a focal region. Furthermore, by using a defocusing-type plane wave as the ultrasound for diagnosis, a shear wave may be observed accurately in a wider area.

It should be understood that the exemplary embodiments described herein should be considered in a descriptive sense only and not for purposes of limitation. Descriptions of features or aspects within each embodiment should typically be considered as available for other similar features or aspects in other embodiments.

Although a few embodiments have been shown and described, it would be appreciated by those skilled in the art that changes may be made in these embodiments without departing from the principles and spirit of the invention, the scope of which is defined in the claims and their equivalents.

What is claimed is:

1. A method of treatment and diagnosis using ultrasound, the method comprising:
inducing a shear wave around a treatment site into which ultrasound for treatment is irradiated, by using the ultrasound for treatment;
irradiating ultrasound for diagnosis into the treatment site;
obtaining a degree of a change in the properties of a tissue of the treatment site by using a displacement of the shear wave measured by an echo ultrasound produced such that the ultrasound for diagnosis is reflected; and
determining a degree of treatment of the tissue of the treatment site based on the degree of a change in the properties of the tissue.

2. The method of claim 1, wherein, in the inducing, the shear wave is induced around each of a plurality of multifocal regions by using the ultrasound for treatment.

3. The method of claim 1, wherein around the treatment site corresponds to a side region of the treatment site with respect to a direction in which the ultrasound for treatment proceeds.

4. The method of claim 1, wherein the ultrasound for diagnosis is a defocusing-type plane wave.

5. The method of claim 1, wherein the ultrasound for diagnosis is irradiated in such a manner that the irradiation of the ultrasound for diagnosis becomes delayed towards sides of an array of an ultrasonic diagnosis device, with respect to a direction in which the ultrasound for diagnosis proceeds.

6. The method of claim 1, further comprising generating ultrasonic images of the treatment site by using the echo ultrasound, wherein the obtaining comprises obtaining a degree of a change in the properties of the tissue by using a displacement of the shear wave measured by using the generated ultrasonic images.

7. The method of claim 6, wherein the obtaining of the degree comprises measuring the displacement of the shear wave by cross-correlating the generated ultrasonic images; and calculating a shear modulus of the tissue of the treatment site by using the measured displacement of the shear wave, wherein the obtaining of the degree comprises obtain a degree of a change in the properties of the tissue, corresponding to a change in the calculated shear modulus.

8. The method of claim 7, wherein the determining comprises determining based on the change in the shear modulus of the tissue that necrosis of the tissue occurs at a time when an infection point, where the elasticity of the tissue increases as a time spent treating the treatment site by using the ultrasound for treatment passes, appears.

9. The method of claim 8, wherein, until it is determined that the necrosis of the tissue occurs, the irradiating of the ultrasound for treatment into the treatment site and the obtaining of the change in the properties of the tissue by the induction of the shear wave are repeatedly performed.

10. The method of claim 1, wherein the change in the properties of a tissue comprises a change in a shear modulus of the tissue.

11. A non-transitory computer readable recording medium storing a program for implementing the method of claim 1.

12. A system for treatment and diagnosis using ultrasound, the system comprising:
an ultrasonic treatment device to irradiate ultrasound for treatment into a tumor site that is to be treated and induce a shear wave around the tumor site by using the ultrasound for treatment;
an ultrasonic diagnosis device to irradiate ultrasound for diagnosis into the tumor site and receive an echo ultrasound produced such that the ultrasound for diagnosis is reflected; and
a processor to obtain a degree of a change in the properties of a tissue of the tumor site by using a displacement of the shear wave measured by the echo ultrasound and determining a degree of treatment of the tissue of the tumor site based on the degree of a change in the properties of the tissue.

13. The system of claim 12, wherein, in the ultrasonic treatment device, the shear wave is induced around each of a plurality of multifocal regions by using the ultrasound for treatment.

14. The system of claim 12, wherein around the tumor site corresponds to a side region of the tumor site, with respect to a direction in which the ultrasound for treatment proceeds.

15. The system of claim 12, wherein the ultrasound for diagnosis is a defocusing-type plane wave.

16. The system of claim 12, wherein the ultrasonic diagnosis device irradiates the ultrasound for diagnosis in such a manner that the irradiation of the ultrasound for diagnosis becomes delayed towards sides of an array of the ultrasonic diagnosis device, with respect to a direction in which the ultrasound for diagnosis proceeds.

17. The system of claim 12, wherein the processor comprises an image generation unit for generating ultrasonic images of the tumor site by using the echo ultrasound and obtains a degree of a change in the properties of the tissue by using a displacement of the shear wave measured by using the generated ultrasonic images.

18. The system of claim 17, wherein the processor further comprises:
a displacement measurement unit to measure the displacement of the shear wave by cross-correlating the generated ultrasonic images;
a calculation unit to calculate a shear modulus of the tissue of the tumor site by using the measured displacement of the shear wave; and
a determination unit to determine whether necrosis of the tissue occurs based on a change in the properties of the tissue, corresponding to a change in the calculated shear modulus.

19. The system of claim 18, wherein the determination unit determines based on the change in the shear modulus of the tissue that the necrosis of the tissue occurs at a time when an infection point, where the elasticity of the tissue increases as a time spent treating the tumor site by using the ultrasound for treatment passes, appears.

20. The system of claim 19, wherein, until it is determined that the necrosis of the tissue occurs, the irradiating of the ultrasound for treatment by the ultrasonic treatment device into the tumor site and the obtaining of the degree of a change in the properties of the tissue by the ultrasonic diagnosis device by the induction of the shear wave are repeatedly performed.
